(19) United States
(12) Patent Application Publication
(10) Pub. No.: US 2015/0045624 A1
(43) Pub. Date: Feb. 12, 2015

(54) Intra-Thoracic Access Device Without Thoracotomy, and Related Methods

(71) Applicant: Minimally Invasive Surgical Access Limited, Dublin 2 (IE)

(72) Inventor: Richard S Stack, Chapel Hill, NC (US)

(21) Appl. No.: 14/453,463

(22) Filed: Aug. 6, 2014

Related U.S. Application Data
(60) Provisional application No. 61/862,940, filed on Aug. 6, 2013.
(60) Provisional application No. 62/028,437, filed on Jul. 24, 2014.

(51) Int. Cl. A61B 17/02; A61B 1/015; A61B 1/07

(52) U.S. Cl. CPC A61B 17/0218; A61B 1/07; A61B 1/015
USPC 600/205; 600/249; 600/245

(57) ABSTRACT

A device for accessing the intra-thoracic space without the need for a thoracotomy or sternotomy includes a cannula having a shaft positionable through a supra-sternal incision into a retrosternal space and a stabilization system for supporting the cannula in a fixed position. The cannula includes illumination features used to illuminate the retrosternal space, giving clear, direct visualization of the retrosternal space during dissection to, and performance of procedures at, the surgical site.

This application claims the benefit of U.S. Provisional Application No. 61/862,940, filed Aug. 6, 2013, and U.S. Provisional Application No. 62/028,437, filed 24 Jul. 2014, each of which is incorporated herein by reference.

### FIELD OF THE INVENTION

The field of the invention relates generally to the field of percutaneous access systems, and more specifically to the field of percutaneous systems for direct percutaneous access to the thoracic cavity without sternotomy or thoracotomy.

### BACKGROUND

Systems and methods for direct percutaneous access to the aorta, such as for treatment of the valve or for implantation of a prosthesis, are described in commonly owned WO 2014/032035, entitled Direct Aortic Access System for Transcatheter Aortic Valve Procedures, which is incorporated hereby by reference. The system disclosed in that application includes a master cannula that, during use, is positioned along the midline of the posterior surface of the sternum so as to provide access for the tools used to gain entry into the aorta. To enter the aorta, a purse-string suture is first placed on the aorta under direct visualization through a scope positioned in the master cannula. An introducer needle is inserted into the master cannula and advanced into the aorta in the center of the purse-string suture, and a guide wire is advanced into the ascending aorta. An introducer sheath is introduced over the guide wire using a dilator and advanced into position in the ascending aorta. The dilator is then removed. Instruments used to carry out the procedure within the aorta are inserted through the introducer sheath. Because the procedure is performed with the heart pumping and blood circulating through the vasculature, the purse string is used to maintain sealing around the introducer sheath.

The present application describes a cannula system that is stabilized and that provides illumination of the working space within the body. By illuminating the aorta and surrounding tissue, the cannula allows the access procedure described above to be carried out without the use of a scope or video assistance, thus optimizing the availability of working space within the cannula and, if a separate scope is to be used, preventing “sword fighting” between the scope and other instruments. The disclosed cannula system is particularly well suited for use in performing surgical procedures within the thoracic cavity without the need for a sternotomy or thoracotomy, including direct aortic access methods disclosed in the prior applications, but may also be used with other systems and procedures for percutaneous access into the body.

### BRIEF DESCRIPTION OF THE DRAWINGS

FIG. 1 is a perspective view of components of an illumination and stabilization system.

FIG. 2 is a side elevation view of the system shown in FIG. 1.

FIG. 3 is a top view of the system shown in FIG. 1.

FIGS. 4-7 are views of the cannula, without the suture anchoring features, in which FIG. 4 is a perspective view, FIG. 5 is a side elevation view, FIG. 6 is a top (anterior) view and FIG. 7 is a bottom (posterior) view.

FIG. 8 is similar to FIG. 4 but shows the cannula with the suture anchoring features.

FIG. 9 is a plan view of the proximal end of the cannula shown in FIG. 8.

FIG. 10 is a proximal perspective view of the cannula, suture anchoring features, and fiber optic cables.

FIG. 11 is a perspective view of components of a second embodiment of an illumination and stabilization system.

FIG. 12 is a side elevation view of the system of FIG. 11.

FIG. 13 is a proximal end view of the system of FIG. 11.

FIG. 14A is a proximal perspective view of an alternative embodiment of a cannula for use in the system of FIG. 1 or 11.

FIG. 14B is a side elevation view of the cannula of FIG. 14A.

FIG. 15A illustrates an instrument adapted for use with the system of FIG. 11.

FIG. 15B is a perspective view of the stop shown in FIG. 15A.

FIG. 15C is a partially exploded perspective view of the instrument stabilization feature shown in FIG. 15A.

FIGS. 16A and 16B are perspective views of the cannula of FIG. 11, showing a portion of the shaft of the instrument of FIG. 15A supported in different positions by the stabilization feature.

FIGS. 17A through 17C are cross-sectional side views of the cannula of FIG. 11, showing a portion of the shaft of the instrument of FIG. 15A supported in different orientations by the instrument stabilization feature.

FIG. 18 is a perspective view of a cannula using an alternative embodiment of an instrument stabilization feature.

FIG. 19A is a perspective view of a camera fixture that may be a feature of the cannula.

FIGS. 19B and 19C show the camera fixture of FIG. 19A mounted on the camera and positioned in stowed and accessible positions, respectively.

FIG. 20 illustrates the stabilization arm and table clamp of the system supporting a cannula above a patient table.

FIG. 21 is perspective view showing the cannula mount and the quick connect feature of the stabilization arm prior to their engagement.

FIG. 22 is a perspective view showing the receiver of the quick connect feature of FIG. 21.

FIG. 23 is similar to FIG. 21 and shows the cannula mount inserted into the receiver and prior to locking of the quick connect using the sliding collar.

FIG. 24 is similar to FIG. 23 but shows the locking collar positioned to capture the cannula mount in the receiver.

### DETAILED DESCRIPTION

### First Embodiment

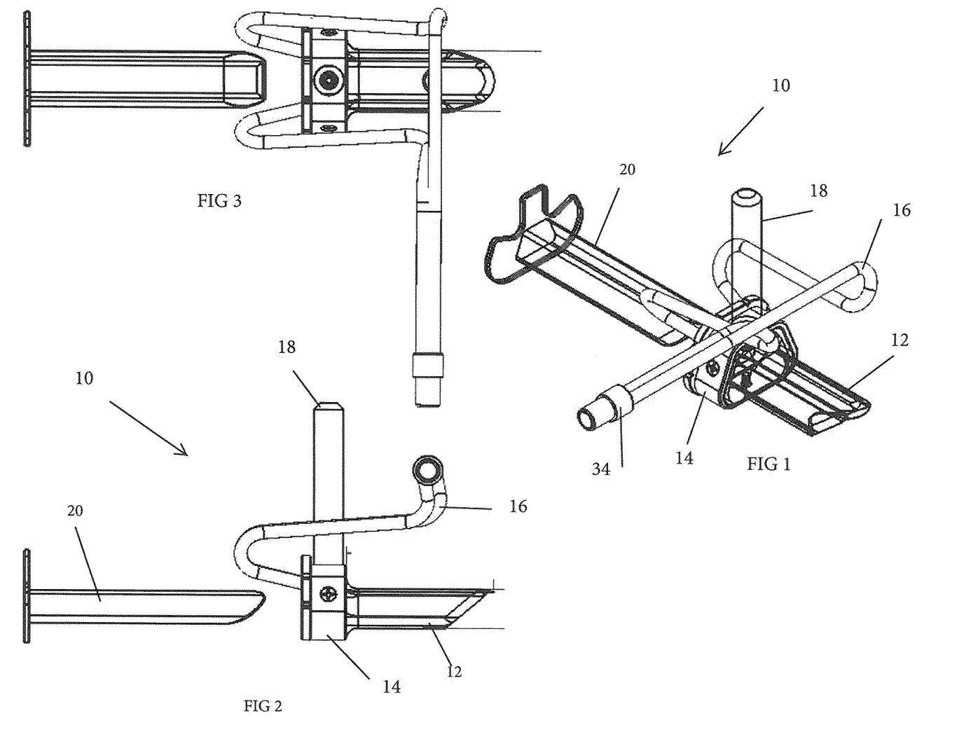

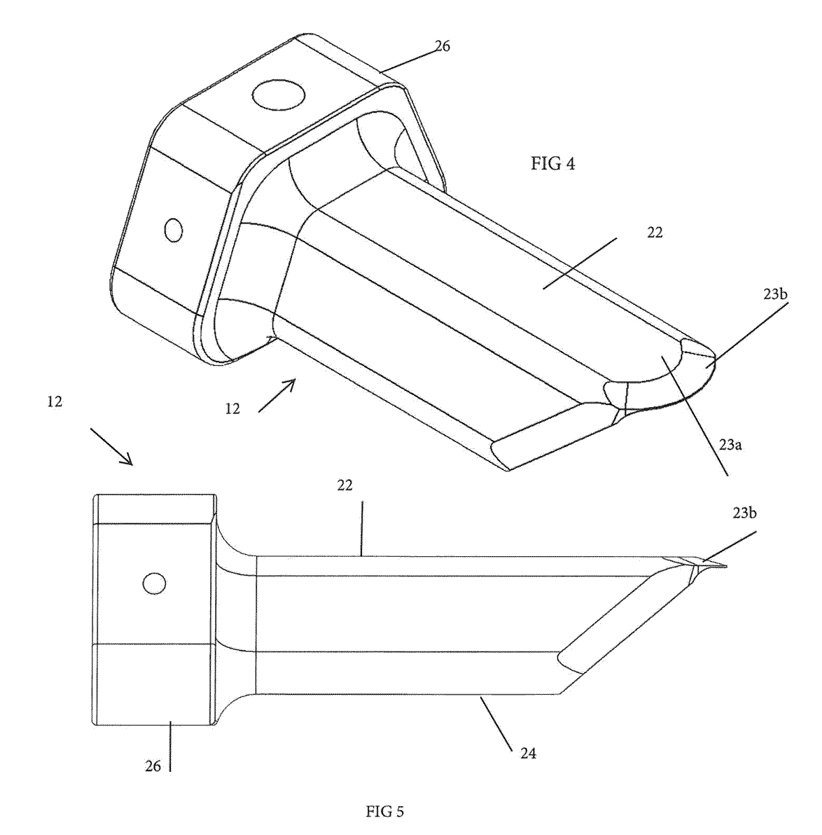

An exemplary illumination and stabilization system 10 is shown in FIG. 1. The system includes an elongate tubular cannula 12 having a lumen extending between its proximal and distal ends. As best seen in FIG. 8, the proximal portion of the cannula 12 defines a flange 26 proportioned to remain outside the body when the tubular shaft of the cannula is disposed through the percutaneous incision, and to optionally seat against the skin surrounding the percutaneous incision. The shaft of the cannula extends distally from the flange 26 and has a generally trapezoidal cross-section as shown.

One or more (two are shown) light cables 16 (e.g. fiber optic cables) are coupled to transmit light from an illumination source into the lumen of the cannula 12. A handle or mount 18 extends transversely from the flange 26 (or, alternatively, the shaft of the cannula 12). The mount is attachable to a stabilization arm 112 (FIG. 20) coupled to the surgical table or to another stable operating room fixture.

An obturator 20 is positionable within the lumen of the cannula 12 in conventional fashion to facilitate advancement of the cannula 12 through an incision.

The cannula functions as an access way through the skin, a dissector, retractor, and a passage to accommodate instruments to be used in the procedure. Referring to FIGS. 4-7, while cannulas with circular cross-sections could be used, cannula 12 preferably has a cross-section that is non-circular so as to include a planar face 22 or flattened region shaped to seat against the posterior face of the sternum when the cannula 12 is disposed behind the sternum. The illustrated cannula has a generally trapezoidal cross-section, with the parallel faces being the anterior and posterior faces 22, 24. The anterior face 22, which seats against the posterior wall of the sternum, is preferably the narrower of the two faces 22, 24 in the lateral direction. The obturator is designed with a trapezoidal tip which is extended on its anterior surface with corners that are rounded in such a manner as to provide a traumatic blunt dissection of the retrosternal tissue while the obturator is in place within the cannula as described in the “Exemplary Methods” section below. The corner regions between each of the cannula's faces are also preferably rounded so as to avoid tissue trauma during the time the obturator is in place or when used for further final blunt dissection under direct visualization after the obturator has been removed.

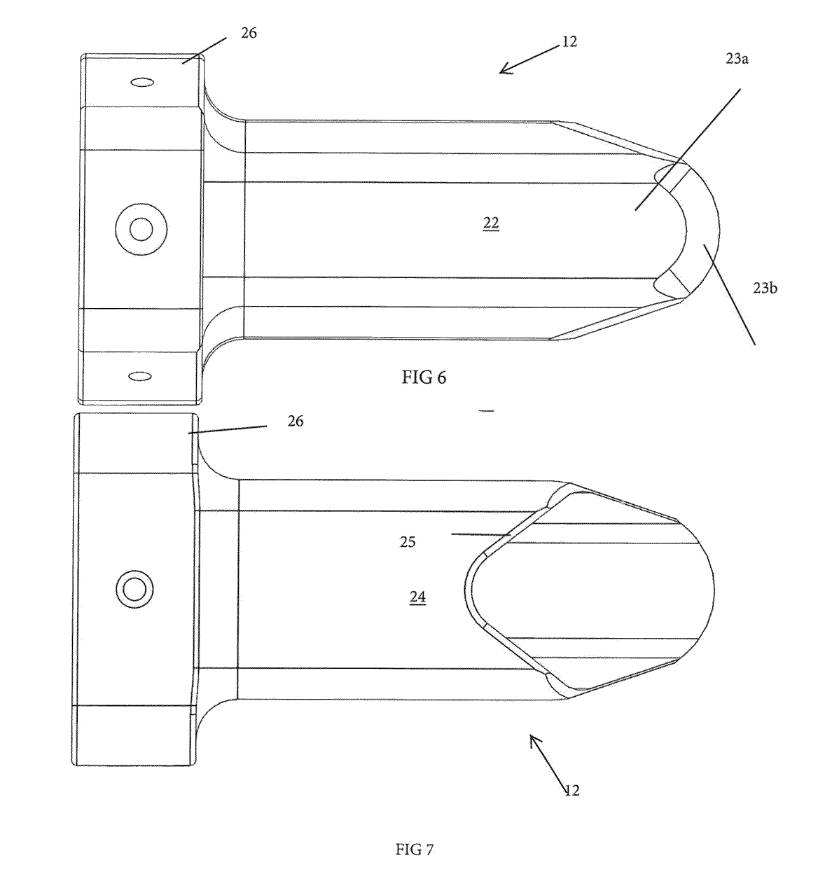

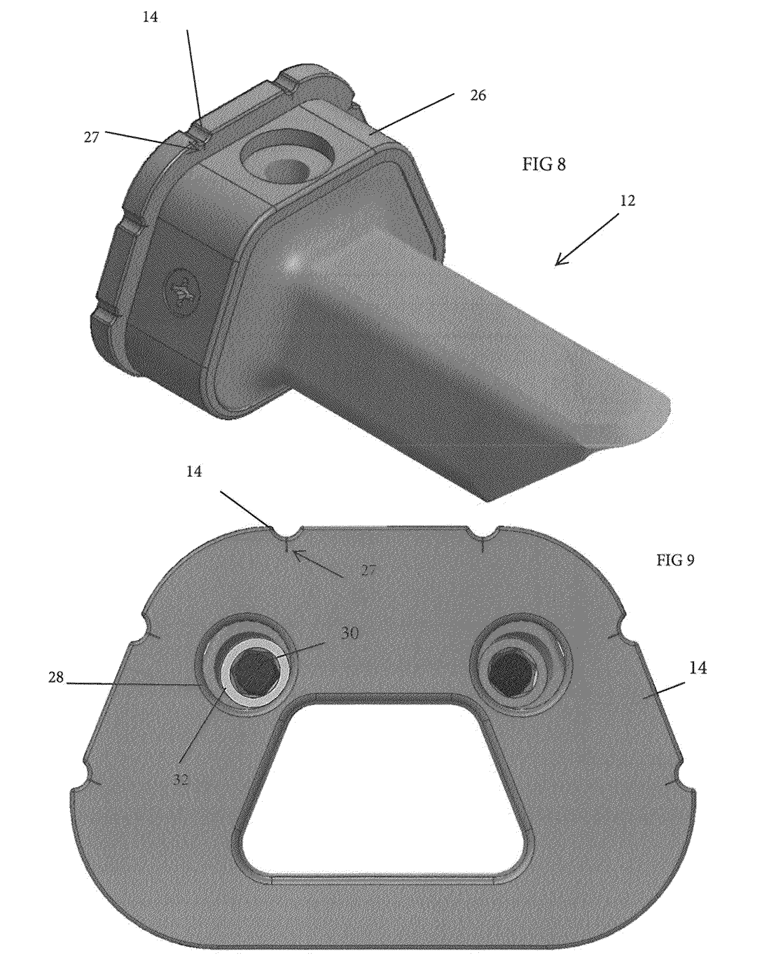

Referring to FIGS. 6 and 7, the anterior face 22 is longer in the longitudinal direction, and thus extends further into the body, than the posterior face 24. It has a distal region in which its width tapers to form a longitudinally extending tongue 23a. Tongue 23a can be used to dissect through tissue and to retract/elevate tissues including fat and vascular structures such as the innominate vein during placement of the cannula through the suprasternal incision into the retrosternal space. A beveled edge 23b on the tongue 23a helps to minimize tissue trauma during insertion of the cannula and elevation of tissues using the tongue 23a. The distal configuration of the anterior surface finally serves as a tissue retractor during the ensuing procedure by maintaining the fully exposed surgical field unobstructed by vascular or other tissues. This fully exposed illuminated surgical field is maintained throughout the procedure in a “hands free” manner by locking the cannula/retractor onto the table mounted stabilization arm 112. This latter feature allows the operator to use both hands necessary for optimal performance of the procedure.

The posterior face 24 of the cannula has a generally V- or U-shaped distal edge 25, with the apex of the V- or U-positioned proximally of the distal end of the cannula 12. This arrangement helps guide instruments passed into the cannula in a posterior direction and thus towards the aorta, and gives the cannula a large distal opening on its posterior side so as to permit passage and manipulation of a valve delivery sheath large enough to accommodate a prosthetic aortic valve, and manipulation of the instruments passed through the cannula (e.g. those used to place the purse string sutures in the aorta) in a posterior direction relative to the distal end of the cannula.

Various materials are suitable for the cannula. In some embodiments, the cannula is rigid, and can utilize a rigid material such as stainless steel. For other embodiments, polymeric materials ranging in hardness from a stiff ABS-like material to a urethane with a hardness in Shore A scale of 40 to 100 may be used. Other suitable materials include PEEK, Delrin, Nylon, Teflon, Polyethelyne, and Polypropylene. The cannula might incorporate a lubricious lining of PTFE or other material. It might be molded or extruded, with additional processing to add other features. Some designs, such as those using Pebax, might be configured to allow the physician to heat the cannula material prior to use to render it deformable, allowing the physician to shape the cannula to a curvature appropriate for the patient. In yet another embodiment, the cannula may incorporate expansion features allowing expansion of the cannula's lumen in one or more directions (e.g. laterally and/or in the anterior-posterior dimension). As one example, expansion features of the type used for vaginal speculums might be used to allow expansion in the desired dimensions.

The flange 26 and any portions of the cannula shaft intended to remain outside the body are preferably formed of a material that is opaque to transmission of visible light.

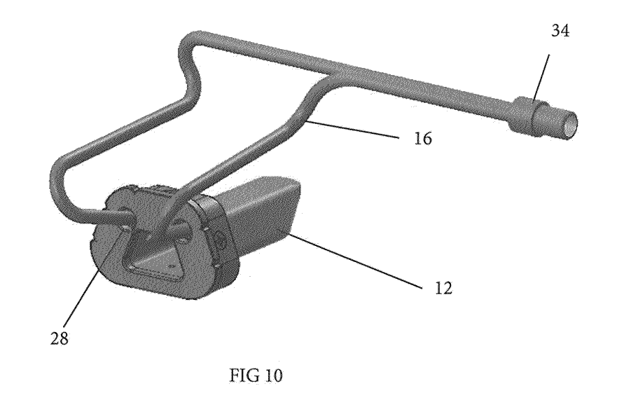

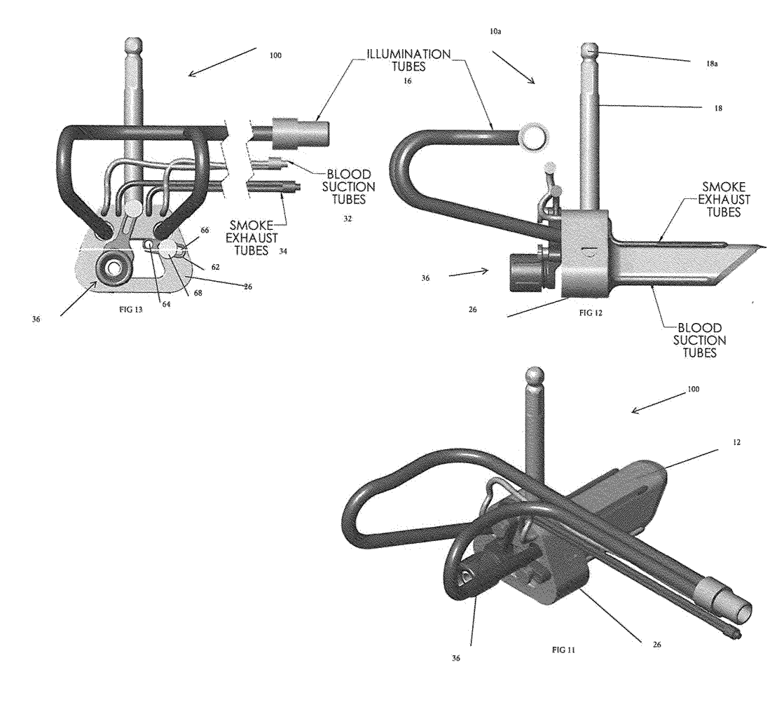

Referring to FIG. 9, flange 26 includes a pair of through-holes 28, each proportioned to receive a fiber optic cable 16 (not shown in FIG. 9, see FIG. 10). The through-holes are aligned with corresponding channels in the wall of the cannula that extend to the lumen of the cannula. A fiber optic tip 30 is disposed in each of the channels and surrounded by an o-ring 32 to minimize leakage of light around its perimeter. The channels angle both inwardly (towards the longitudinal axis of the cannula) and distally such that light transmitted by the fiber optic cables and the tip 30 is directed into the lumen of the cannula and out the distal opening of the cannula.

Fiber optic cables 16 preferably terminate at a common proximal fitting 34 that allows optical coupling of the cables 16 to an illumination source. When coupled to an illumination source, the cables 16 and fiber optic tips 30 transmit light into the interior of the cannula via channels through the body of the cannula, which have distal openings in the lumen of the cannula. The channels within which the optical tips are disposed are preferably oriented such that the cones of light transmitted by the optical tips converge outside the distal opening of the cannula so as to flood the surgical field with light, producing a highly illuminated surgical field, although in alternative embodiments the cones of light might converge within the cannula. In use, the light transmitted by the fiber optic system serves to illuminate the tissues distal to the cannula, including the aorta, allowing the user to more readily apply the purse-string to the aorta and to insert a needle through the tissue bounded by the purse string so as to gain access to the aorta.

Suture anchoring features may be optionally positioned on the proximal end of the cannula. In this embodiment, the suture anchoring features take the form of a plurality of notches 14 formed around the perimeter of the flange 26. Each notch 14 is continuous with a slit 27 as shown. During formation of the purse-string suture as described above, strands of suture may be tucked into the slits where they will remain anchored until removed from the slits.

### Second Embodiment

A second embodiment of an illumination and stabilization system 10a is shown in FIGS. 11 through 19C. As with the first embodiment, the illumination and stabilization system includes an illuminated cannula 12, a stabilization arm 112 (FIG. 20) attachable to the illuminated cannula 12 for supporting the illuminated cannula 12 in a user-selected stable position and orientation relative to the patient, and a clamp or other mounting feature 102 for mounting the stabilization arm 112 to the patient table or to a separate operating room fixture. It should be noted that certain of the features of the system are omitted from certain drawings to allow others of the features to be more easily seen.

Many details of the illuminated cannula 12, including its shape, dimensions etc are set forth in the discussion of the first embodiment and will not be repeated here. As with the cannula of the first embodiment, the cannula functions as an access way through the skin, a dissector, a retractor, and a passage to accommodate instruments to be used in the procedure, and it also provides a stable platform for supporting certain ones of those instruments.

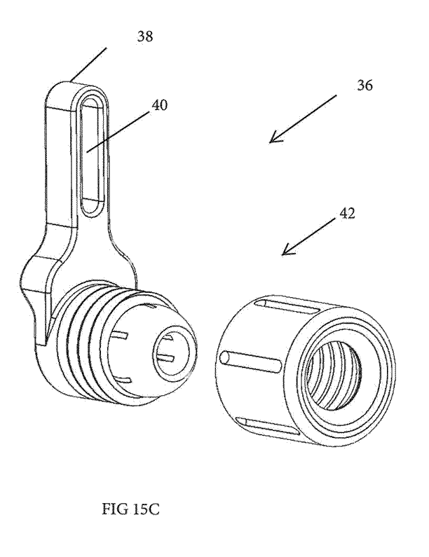

FIG. 14 shows an alternative shape for the cannula in which an enlarged chamber 12a is disposed between the flange 26 and the tubular shaft of the cannula 12. The chamber 12a is defined by a bulge in cannula wall 22 that extends away from the wall 24, giving the cannula lumen a larger diameter at its proximal end and thus creating working space within the cannula. This additional working space helps prevent tight clustering of instruments (particularly rigid instruments) in the proximal part of the cannula. It also gives the proximal shafts of such instruments more room to move when the distal ends of the instruments are pivoted into the apex defined by the U- or V-shaped edge 25 (FIG. 7) of the cannula's posterior wall.

In the second embodiment, the cannula is equipped with fluid/blood aspiration tubes 32 and smoke evacuation tubes 34 extending from the proximal face of the flange 26 or from another part of the flange or cannula 12. These tubes including fittings allowing them to be connected to suction and evacuation systems within the operating room. Each of the tubes 32, 34 is coupled to or integral with corresponding suction/evacuation tubes having distal openings positioned within the lumen of the cannula 12 or along the exterior surface of the cannula 12. During placement of the cannula, which includes cauterization of small bleeding vessels along the dissection plane, these tubes help maintain a clear visual field by drawing smoke and blood out of the body.

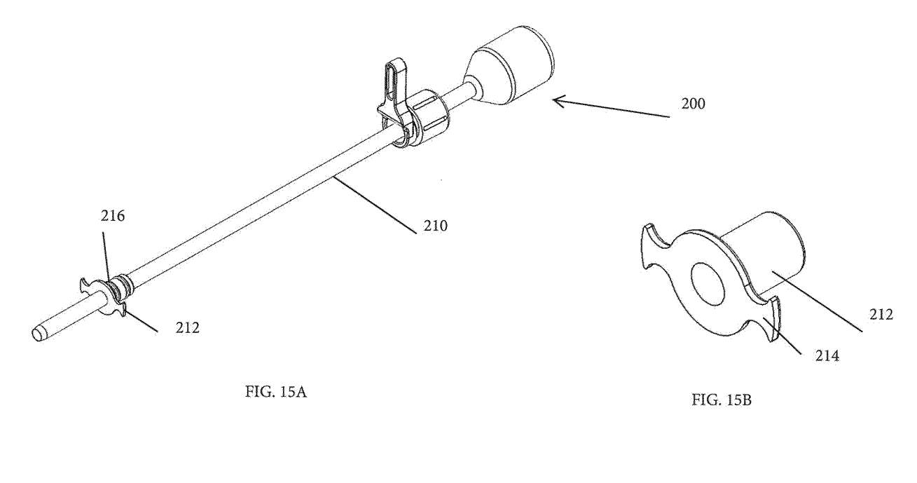

The illumination and stabilization systems are designed to give access to and stable support for certain instruments intended to be passed through the cannula 12 during use of the system. One such instrument is a valve delivery sheath 200 (FIG. 15A) which can be used to deliver a replacement heart valve (e.g. an aortic or mitral valve) to the diseased natural valve site via the aortic access approaches described here.

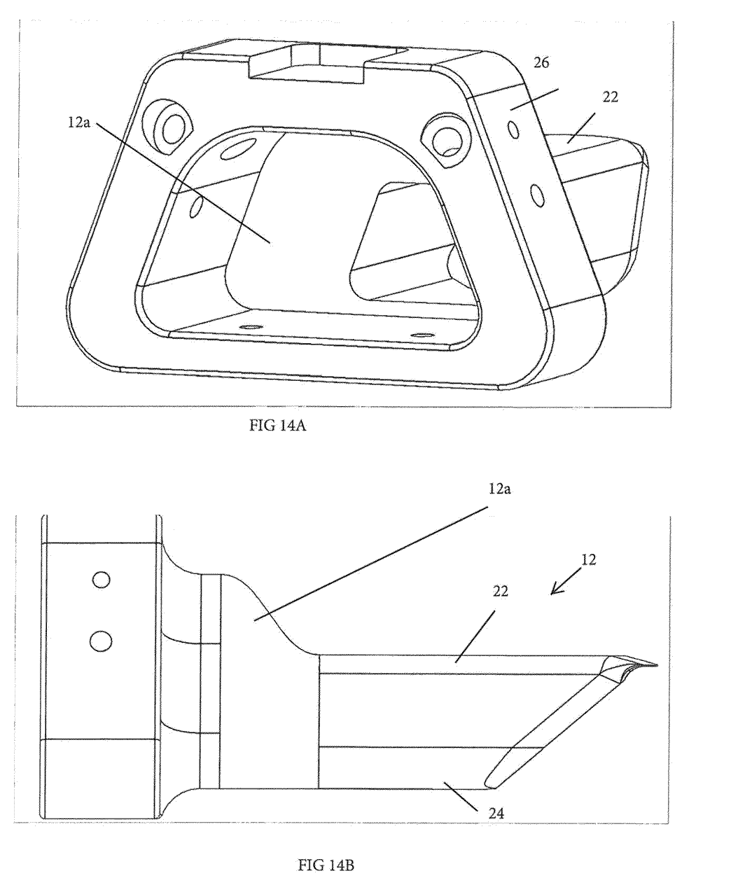

As shown in FIG. 12, cannula 12 includes an instrument stabilization feature 36 at its proximal end, preferably on the flange 26. The stabilization feature 36 lets the user secure an instrument (e.g. sheath 200) to the body of the cannula, which is itself stable due to its connection to the stabilization arm 112 (FIG. 20). The stabilization feature 36 is coupled to the shaft of the delivery sheath either during the manufacturing process or in the procedure room in preparation for surgery. It allows multi-directional positioning of the sheath relative to the cannula, and it can be locked to securely retain the delivery sheath in the user-selected position and orientation. This eliminates the need to have a member of the surgical staff hold the delivery sheath during the valve-delivery procedure.

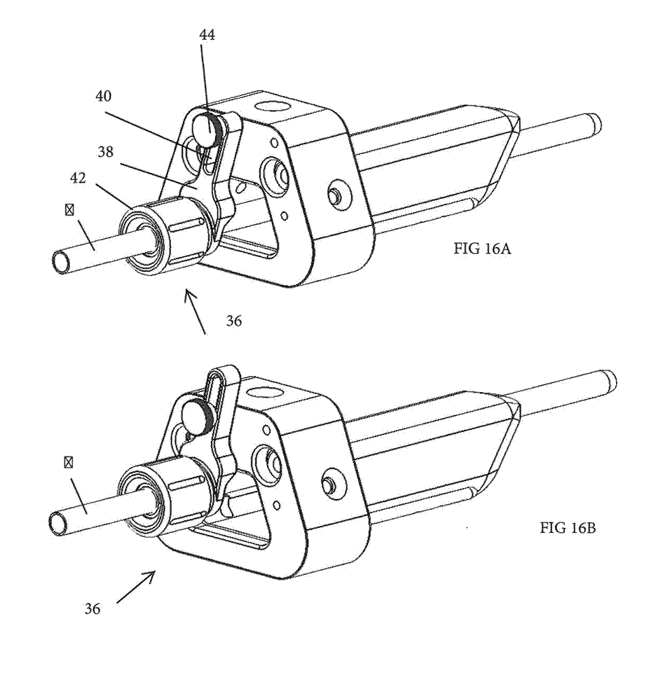

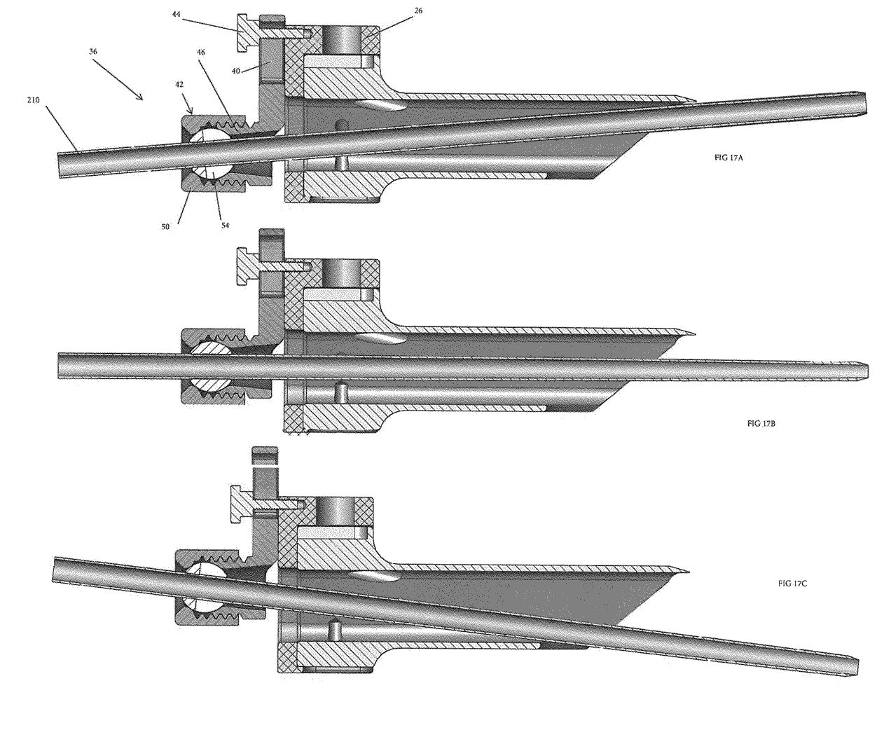

One aspect of the stabilization feature 36 allows the user to select the two-dimensional position of the proximal part of the shaft 210 relative to the proximal opening into the cannula. In other words, if the proximal opening of the cannula is considered to a horizontal axis X and a vertical axis Y that intersect at the center of the proximal opening when view as in FIG. 13, the stabilization feature 36 lets the user select the X- and Y-positions of the shaft at the proximal opening. See, for example, FIG. 16A in which the sheath's shaft 210 passes into the proximal opening a location that is low along the Y axis and towards the left of the X axis, and FIG. 16B in which the sheath's shaft 210 crosses into the proximal opening at a location that is high along the Y axis and towards the left of the X axis. As shown in these drawings, the stabilization feature 36 includes a member 38 having an elongate slot 40 and a coupling 42 designed to couple to the delivery sheath's shaft. A set screw 44 extends through the slot to connect the member 38 to the flange 26 of the cannula 12. To adjust the X, Y position of the delivery sheath at the proximal opening, the user loosens the set screw 44, and then repositions the delivery sheath by sliding the member 38 relative to the set screw 44 (compare the positions of the set screw 44 and slot 40 in FIGS. 16A and 16B) and/or rotates the member 38 relative to the set screw 44. When the delivery sheath 200 is in the desired position at the proximal opening the set screw 44 is tightened to fix delivery sheath's position at the proximal opening.

A second aspect of the stabilization feature 36 allows the user to pivot the delivery sheath 200 relative its point of connection to the stabilization feature 36, and a third aspect of the stabilization feature 36 allows the user to adjust the longitudinal (or “Z-axis”) position of the delivery sheath relative to the cannula 12. Referring to the exploded view of FIG. 15C, coupling 42 has a first threaded part 46 (shown as a male threaded part having a socket 48) and second threaded part 50. The second threaded part 50 is shown as a cap or cup having internal threads engaged with those of the male threaded part, and also including a proximal opening 52. A hollow spherical ball 54 is captured between the first and second threaded parts 46, 50. Coaxial proximal and distal apertures 56 (only the proximal one is visible in FIG. 15C) are positioned to receive the shaft 210 so that it extends through the spherical ball 54. The ball includes engagement features 58 defined by longitudinal slots 60 extending proximally from the distal aperture. As shown in FIGS. 17A through 17C, the coupling 42 receives the shaft 210 through the opening 52, aperture 56, and socket 48.

The threaded parts 46, 50 have a first, “loosened” position in which the spherical ball 54 can rotate within the threaded parts of the coupling 42, and a second “tightened” position in which the positioning of the spherical ball 54 is fixed relative to the threaded parts of the coupling. When in the “tightened” position, the ball 54 is clamped between the threaded parts 46, 50. Compression of the ball 54 against the socket 48 compresses the ball's engagement features 58 radially inwardly and causes them to frictionally engage the shaft 210, preventing its longitudinal movement.

FIGS. 17A through 17C show the shaft 210 in three different pivotal orientations resulting from angular movement of the proximal end of the delivery sheath relative to the cannula 12 when the threaded parts were in the loosened position. Note that in these figures, the member 38 is also shown three different positions relative to the set screw 44, although it should be appreciated that adjustments at the set screw 44 and at the spherical ball 54 may be made independently.

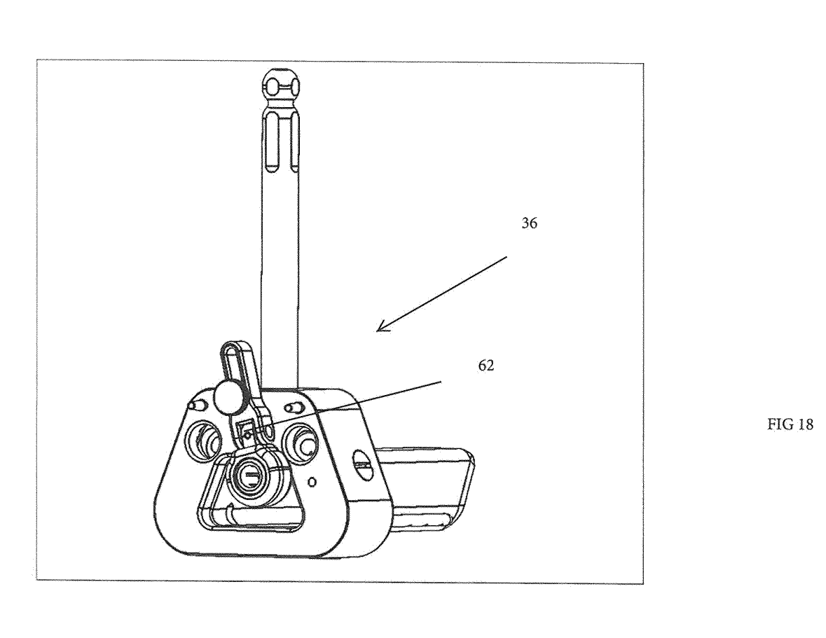

FIG. 18 shows a modified version of the coupling 42, in which a hinge 62 is positioned between the member 38 and the coupling 42. This allows the coupling 42 to be pivoted out of alignment with the proximal opening of the cannula, thus leaving the opening unobstructed during use of instruments that do not require stabilization using the stabilization feature 36.

As will be described in further detail in the “Method” section below, a stop 212 positioned on the shaft 210 of the valve delivery sheath 200 has wings 214 extending laterally from the shaft 210 that will contact the area of the aortic wall surrounding the incision as the sheath 200 is inserted, creating a barrier to insertion of the sheath beyond the intended depth. During use the user may choose to tether the stop to the purse string sutures bounding the penetration site into the aorta so as to also prevent inadvertent withdrawal of the sheath 200 from the incision site.

The stop is formed of a flexible polymeric material or other suitable material. The delivery sheath 200 may come prepackaged with the stop 212 on its shaft, or the user may thread the stop 212 onto the shaft 210 and slide it along the shaft to a desired position. Suture strands 216 may be wrapped around the barrel of the stop 212 as shown in FIG. 15A to secure it on the shaft 210.

Because the illumination features of the cannula give clear direct visualization of the retrosternal space and aorta, video assistance of the aortic access procedure is not needed for carrying out the procedure. However, the surgical team may wish to capture images of the procedure for teaching purposes, or to allow students or members of the team who do not have a direct line of sight into the cannula to observe the procedure. The system may include a camera fixture that can support a camera extending through the cannula but that can be moved out of alignment with the proximal opening of the cannula when not in use.

Referring to FIG. 13, a first camera fixture 62 includes a member having an aperture 64 through which a camera may be positioned, a slot 66, and a set screw 68 extending through the slot 66. The positioned of the aperture relative to the proximal opening of the cannula is adjusted by loosening the set screw 68 and then sliding the fixture 62 relative to the set screw 68 and/or rotating the fixture 62 about the set screw.

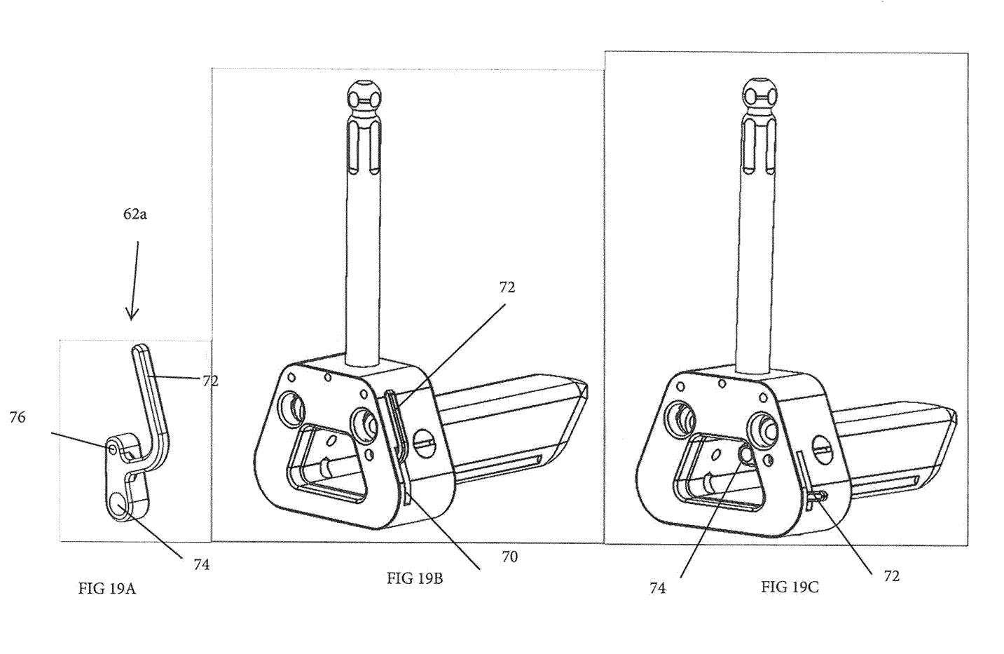

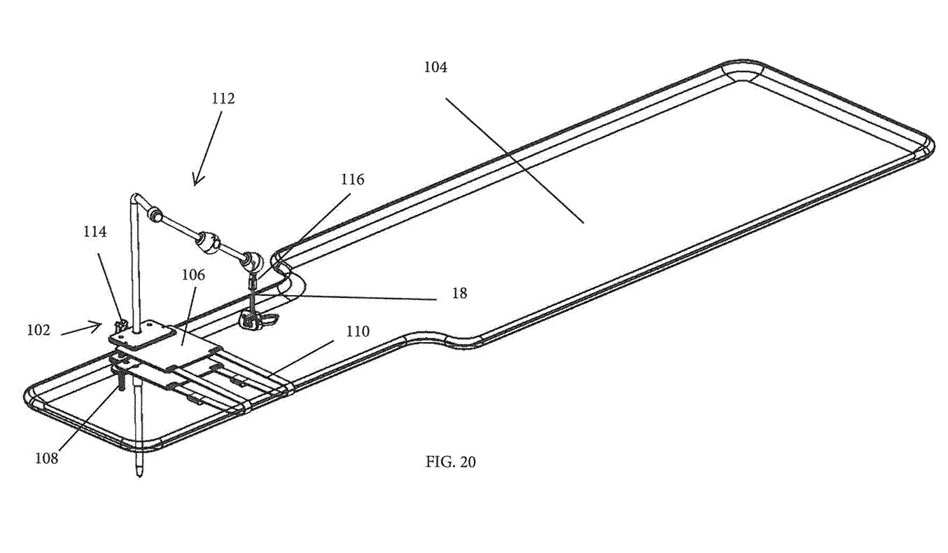

An alternative camera fixture 62a is shown on its own in FIG. 19A, and as assembled with the cannula in FIGS. 19B and 19C. Fixture 62a is disposed within a slot 70 in the flange 70. It includes a lever 72 partially extending from the slot 70, and an aperture 74 on the opposite side of a pivot point 76 from the lever 72. The lever 72 pivots relative to a pin in the aperture 76 between a first position (FIG. 19C) in which the aperture 74 extends into the proximal opening of the cannula, and a second, stowed, position in which the aperture is recessed into the slot 70.

Referring again to FIG. 20, a stabilization arm 112 is used to maintain the cannula in a stable position relative to the operating table 104. Since the radiolucent tables used for procedures conducted using fluoroscopy are generally provided without rails, a clamp 102 is designed to clamp against the table 104 itself rather than attach to rails on a table. In particular, the clamp 102 includes a pair of radiolucent plates 106 on one or more threaded rods. The plates are positionable against opposite surfaces of the table 104. The plates are supported on one or more threaded rods 108 that are used to move the plates towards one another and then locked in place so as to clamp onto the table. Straps 110 are tightened laterally around the table to further stabilize and secure the clamp 102. The stabilization arm is attached to the clamp 102. A preferred stabilization arm 112 includes a plurality of links and locking joints. A locking mechanism which may be a knob 114 is rotated to simultaneously disengage and reengage the locks of the joints for reorientation of the cannula. In some embodiments, the entirety of the mount and stabilization arm may be made of radiolucent materials.

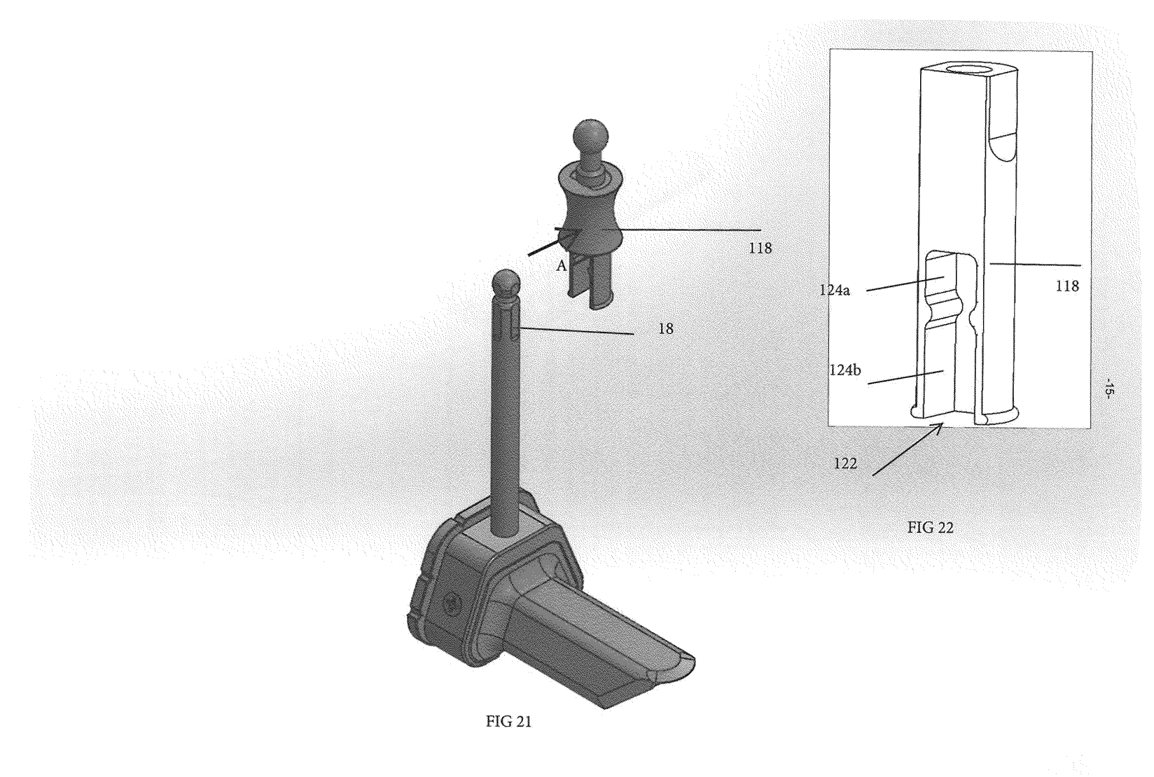

A quick connect feature 116 is used to couple the cannula to the stabilization arm 112. The quick connect 116 is positioned on the stabilization arm 116 and is designed engage the mount 18 of the cannula. As best shown in FIG. 21, quick connect 116 includes receiver 118 and a sliding collar 120 disposed around the receiver 118. The top of the receiver is designed to be attached to the stabilization arm prior to the procedure, using the ball mechanism shown in FIG. 21 or an alternate mechanism such as a threaded arrangement.

FIG. 22 shows receiver 118 separate from the other components of the quick connect feature 116. Receiver 118 has a non-circular outer cross-section such as the D-shaped cross-section shown in the drawings. A lateral opening 122 in the receiver includes upper and lower portions 124a, 124b separated by a waist. These portions 124a, 124b correspond to upper and lower portions 128a, 128b of the mount 18. The relative cross-sectional shapes of the lateral opening 122 of the receiver 118 and those of the upper and lower portions 128a,b of the mount 18 are are selected to allow insertion of the upper and lower portions of the mount 18 into the upper and lower portions of the receiver, respectively, to key the mount 18 and receiver 118 together to prevent rotation of the mount relative to the receiver 118. While other cross-sectional shapes are available, the mount 18 has a hex head as its upper portion 128a and a shaft with planar faces as its lower portions 128b, and the cross-sectional shape of the lateral opening 122 is square. These shapes allow the mount 18 to be laterally inserted into the opening 122 as indicated by arrow A in FIG. 21, and prevent rotation of the mount 18 within the receiver.

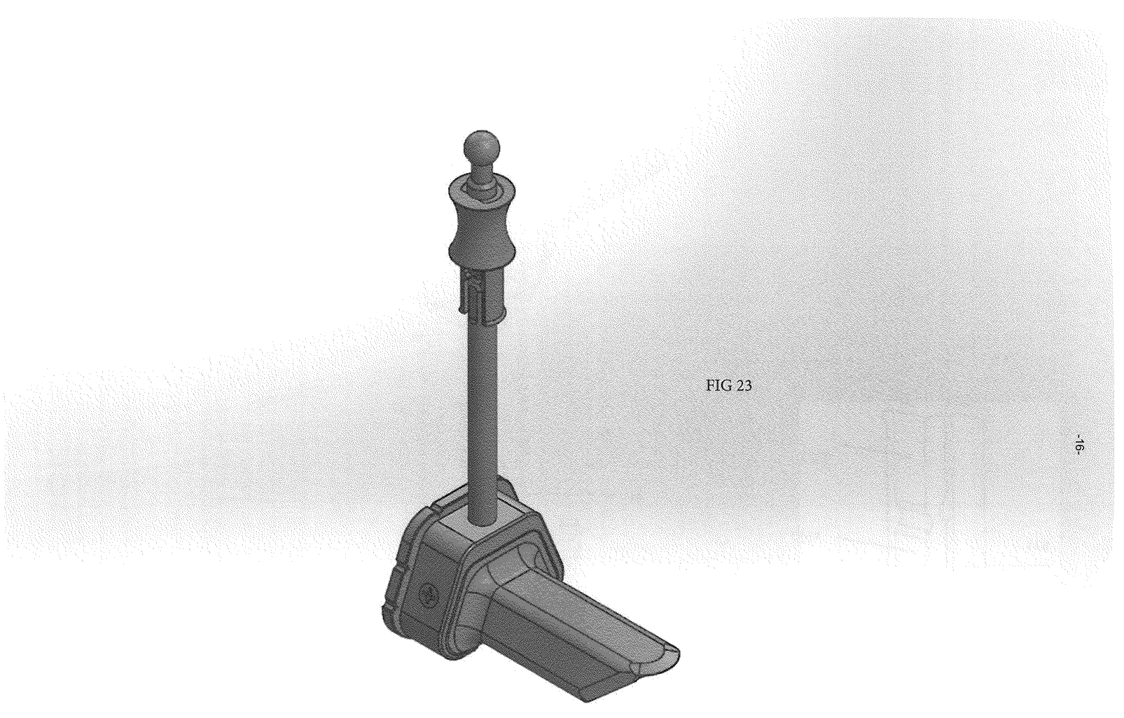

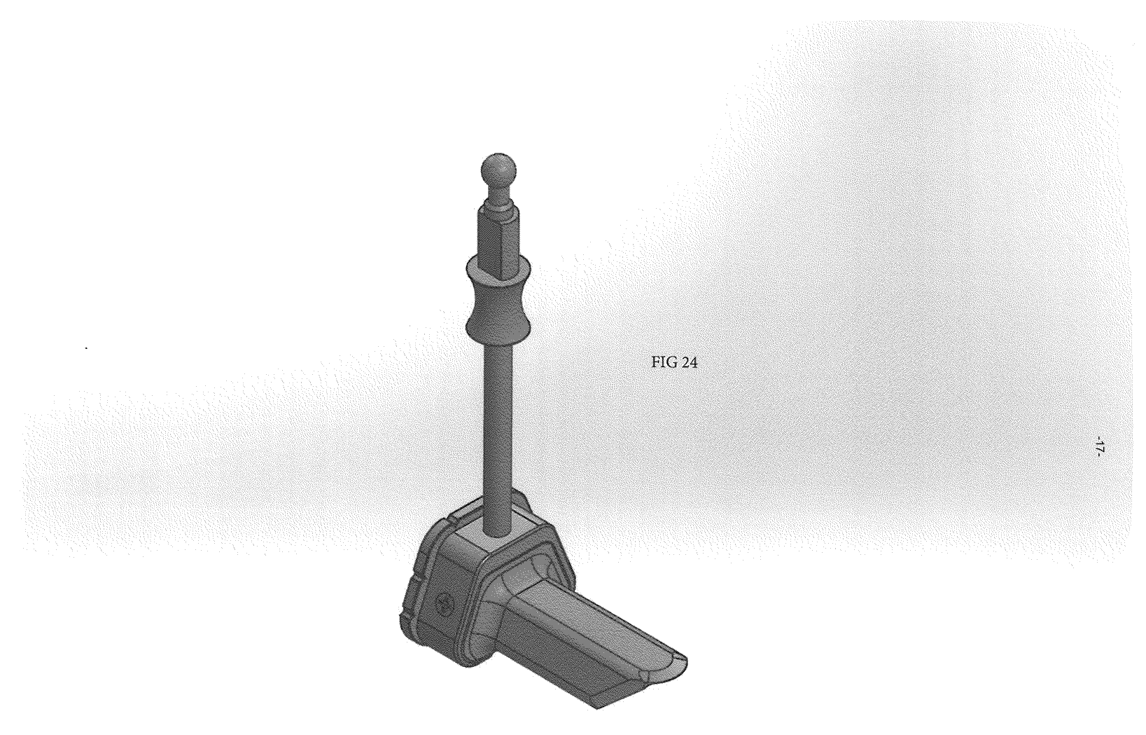

Sliding collar 120 is longitudinally slidable over the receiver from a first, withdrawn, position shown in FIGS. 21 and 23 and a second, extended, position shown in FIG. 24 which captures the mount within the opening 122. The collar has a non-circular lumen (D-shaped in the drawings) including a planar wall that closes the opening 122 so as to aid in preventing rotation of the mount 118.

### Exemplary Method

The following section describes steps of an exemplary method for percutaneous access of the aorta using the system described herein without the need for a thoracotomy.

A 2.5 cm skin incision is made with a #15 blade 5-7 mm above the sternal notch in the midline in a semi-circle (collar) fashion. Cautery is use to extend the incision between the sternocleidomastoid muscles to the level of the sternohyoid muscles. The midline fat plane between the sternohyoid muscles inferior to the thyroid isthmus is entered and extended in a vertical fashion to the level of the pretracheal fascia enabling the sternohyoid and omohyoid to be liberated laterally.

From the patient's right side, the surgeon uses his/her left index finger in a supinate position to identify the trachea. The finger is used to bluntly dissect inferiorly adjacent and posterior to the sternum into the mediastinum until the aorta and innominate artery are digitally identified.

Blunt dissection is redirected when the aorta and innominate artery are palpated by pronating the left index finger and developing the plane just to the cephalad portion of the aortic arch and the origin of the innominate artery.

At this point, the finger is removed and the cannula 12 with the obturator 20 in place is positioned gently but firmly into the space anterior to the innominate artery just at the thoracic inlet behind the sternal notch and between the strap muscles of the neck.

The device is “quick-connected” to the stabilization arm 112 that is strongly mounted to the procedure table. This is done by inserting the mount 18 into the receiver 118 and lowering the sliding collar 120 to capture the mount 18.

The joints of the stabilization arm 112 are tightened and secured while elevating/lifting the manubrium with the anterior surface 22 of the cannula 12 against the back of the sternum while it assumes a position aimed at the final surgical field that will center upon the aorta or the innominate artery with the middle of the cannula's distal opening and the cleft 25 of the posterior surface 24 of the cannula 12. The prevascular plane of the innominate artery which is easily seen (after illumination has begun using the illumination cables) through the open end of the cannula following removal of the obturator is liberated and extended towards it's origin and the aorta using scissors, forceps, and cautery. As the anterior prevascular plane is being developed, the anterior mediastinal fat and the innominate vein are liberated from the aorta and innominate artery.

The stabilization arm 112 is loosened allowing free movement of the device which is repositioned in a manner that allows it to be further encouraged deeper into the mediastinum under direct, illuminated observation, anterior to the aorta and innominate artery but posterior to the mediastinal fat and the innominate vein.

The specially designed anterior surface 22 of the distal end of the cannula 12, with the extended, beveled “tongue” 23a, 23b is then utilized to elevate the innominate vein and mediastinal fat from the aorta and innominate artery by placing the tongue extension behind the vein and fat. While these structures would normally block visualization of an appropriate aortic access site, they are lifted out of the way as the device is further advanced to a position exposing the ascending aorta adjacent to the origin of the innominate artery and fully retracting the left innominate vein out of the way of the surgical field for the duration of the procedure. Blunt and sharp dissection provides visualization and assistance to generate optimal exposure of the ideal access site for the aorta behind the innominate vein. This unique route of access to the aorta goes between and avoids entry into either pleural cavity, thus avoiding the need for a chest tube which would otherwise accompany any conventional thoracotomy route that would unavoidably require penetration of the chest wall and creation of a pneumothorax.

As noted, during formation of the desired retrosternal surgical dissection plan and placement of the cannula, the light cable on the cannula 12 is connected to a standard 300 W Xenon light source for optimal continuous illumination of the surgical field. The cannula's continuous suction ports and the smoke evacuator ports are also connected to the OR suction tubes.

The stabilization arm 112 is finally firmly locked into place providing a stable and secure, fully retracted and illuminated surgical field for the duration of the procedure. This system now allows the surgeon to operate “hands free” from the access system, freeing up both hands for the remainder of the operation.

Pursestring sutures are then applied to the illuminated wall of the aorta in preparation for penetration of the aortic wall with a vascular sheath. Placement of the pursestring is optimized by the unique shape and dimensions of the cannula 12. On the distal end the trapezoidal shape of the cannula 12 places the greatest lateral dimension at the posterior surface of the device which includes the U- or V-shaped notch at wall 25 and which is sculpted to permit maximum exposure of the surgical field and to permit maximum excursion of the rigid surgical instruments used in formation of the pursestring without the “sword fighting” interference of one instrument with the other as would occur with a conventional tube. The proximal mouth of the cannula (FIG. 14) is greatly expanded compared to the portion that enters the patient's body to allow maximum angular excursion of the surgical instruments.

An introducer needle is then inserted into the aorta in the center of the pursestrings and, after confirming blood return, a soft tip guidewire is positioned through the introducer needle and the introducer needle is removed. Ultimately, valve delivery sheath 200 is passed into the aorta over a guidewire disposed within the needle puncture. The stop 212 gives tactile feedback when it reaches the wall of the aorta and prevents further advancement of the sheath 200. The pursestring sutures may be secured around the stop 212—thus also preventing the sheath 200 from pulling out of the aorta. The instrument stabilization feature 36 is attached to the cannula 12 using set screw 44.

The valve replacement is carried out, after which the delivery sheath 200 is removed and the pursestring sutures tied securely.

In a slightly modified variation of this method, the pursestring sutures and needle puncture are formed in the illuminated innominate artery and used to gain access into the innominate artery, and instruments (e.g. delivery sheath) subsequently passed into the innominate artery are directed to the aorta.

While this exemplary method is described in the context of entry into the aorta, it is equally suitable for other procedures to be performed in the thoracic cavity, allowing such other procedures to be formed without thoracotomy or sternotomy and without entering either pleural cavity. Additionally, procedures to be performed elsewhere in the heart, including those for mitral valve treatment or replacement, may be carried out using access to the aorta using the procedures described here.

All patents and applications referred to herein, including for purposes of priority, are incorporated herein by reference.

We claim:

1. A cannula system comprising:
an elongate cannula having a proximal end, a distal end, and a lumen extending between the proximal and distal ends; and
at least one fiber optic cable couplable to an illumination source, the fiber optic cable attachable to the cannula so as to transmit light from the illumination source into the lumen such that said light illuminates structures distal to the distal end of the cannula.

2. The cannula system of claim 1, wherein the cannula includes a side wall, and an opening through the side wall of the cannula, and wherein the fiber optic cable is attachable to the cannula at the opening.

3. The cannula system of claim 1, wherein the cannula includes at least one anterior wall having a generally planar surface positionable in contact with a posterior surface of a sternum.

4. The cannula system of claim 3, wherein the cannula has a generally trapezoidal cross-section.

5. The cannula system of claim 4, wherein the cannula includes a plurality of longitudinally-extending walls, including the anterior wall, a posterior wall parallel to the anterior wall, and a pair of side walls extending between the anterior and posterior walls.

6. The cannula system of claim 5, wherein the width of the anterior wall is narrower than the width of the posterior wall.

7. The cannula system of claim 6, wherein the anterior wall has a distal edge distal to the distal-most edge of the posterior wall.

8. The cannula system of claim 7, wherein the posterior wall has a generally V- or U-shaped distal edge, the apex of the V- or U-shaped edge extending in a proximal direction.

9. The cannula system of claim 1, further including a plurality of suture anchoring elements positioned on a proximal portion of the cannula.

10. The cannula system of claim 1, wherein the cannula includes a mount and wherein the system includes a stabilization arm attachable to the mount.

11. The cannula system of claim 10, wherein the stabilization arm is attachable to a patient table.

12. The cannula system of claim 11, further including a clamp having radiolucent first and second plates positionable against first and second surfaces of the patient table to engage the table.

13. The cannula system of claim 12, wherein the clamp includes at least one strap extendable around the patient table in a lateral direction from the first plate to the second plate.

14. The cannula system of claim 1, further including an instrument stabilization feature coupled to the cannula, the stabilization feature configured to stabilize a shaft of an instrument extending through the lumen of the cannula.

15. The cannula system of claim 14 wherein the stabilization feature includes an unlocked position in which the instrument is moveable relative to the cannula and a locked position in which the instrument is restrained against movement relative to the cannula.

16. The cannula system of claim 1 further including a camera fixture positioned on the camera.

17. The cannula system of claim 1, wherein the cannula includes at least one of a smoke evacuation port and a fluid suction port.

18. The cannula system of claim 7, wherein the distal edge has a tapered lateral dimension to form a tongue.

19. The cannula system of claim 18 wherein the tongue includes a beveled distal tip.

20. A method of percutaneously accessing a thoracic cavity without a sternotomy or thoracotomy, comprising the steps of:
forming an incision above a supra-sternal notch;
inserting a cannula having a lumen through the incision into a retrosternal space;
coupling a light source to light cables carried by the cannula;
illuminating the light source to illuminate a surgical field within the thoracic cavity;
mounting the cannula to a stabilization arm; and
performing a procedure in the illuminated surgical field.

21. The method of claim 20, wherein the cannula includes a longitudinally extending tongue and wherein the method includes retracting tissue in the retrosternal space using the tongue.

22. The method of claim 21, wherein retracting tissue includes retracting an innominate vein.

23. The method of claim 20, wherein the method includes dissecting tissue in the retrosternal space using the cannula.

24. The method of claim 20, further including evacuating smoke or blood from the thoracic cavity through evacuation lumen carried by the cannula.

25. The method of claim 24, further including inserting an instrument through the lumen for use in performing a procedure at the surgical site, coupling the instrument to a stabilization feature on the cannula, placing the stabilization feature in an unlocked position and repositioning the instrument relative to the cannula, and placing the stabilization feature in a locked position to fix a position of the instrument relative to the cannula.

26. The method of claim 20, wherein light cables produce cones of light converging at the surgical site.

27. The method of claim 26, wherein the light cables have distal ends at the lumen, and wherein illuminating the light source causes the cones of light to be directed through the lumen to the surgical site.

26. The method of claim 25 wherein the procedure is a valve implantation procedure, and wherein the instrument is a valve delivery sheath.

27. The method of claim 20, wherein performing the procedure includes passing instruments between the pleural cavities without entering the pleural cavities.
